(19) United States
(12) Patent Application Publication
(10) Pub. No.: US 2014/0309477 A1
(43) Pub. Date: Oct. 16, 2014

(54) CT ATLAS OF THE BRISBANE 2000 SYSTEM OF LIVER ANATOMY FOR RADIATION ONCOLOGISTS

(71) Applicants: H. Lee Moffitt Cancer Center and Research Institute, Inc., Tampa, FL (US); University of South Florida, Tampa, FL (US)

(72) Inventors: Steven Eric Finkelstein, Scottsdale, AZ (US); Sarah E. Hoffe, Bradenton, FL (US); Mark S. Russell, Sherman, TX (US)

(21) Appl. No.: 14/315,107

(22) Filed: Jun. 25, 2014

Related U.S. Application Data
(63) Continuation-in-part of application No. 13/234,903, filed on Sep. 16, 2011.
Application No. 13/234,903 is a continuation of application No. PCT/US2010/027470, filed on Mar. 16, 2010.
(60) Provisional application No. 61/160,396, filed on Mar. 16, 2009.

(51) Int. Cl. A61B 19/00; A61N 5/10; G06T 7/00

(52) U.S. Cl. CPC A61B 19/52; G06T 7/0012; A61N 5/1039; A61B 2019/5289
USPC 600/1

(57) ABSTRACT

The method includes the steps of obtaining atlas data in an atlas coordinate set from a computer-readable atlas of hepatic anatomical information including three orders of division and obtaining patient data in a patient coordinate set. The method further includes morphing atlas data from the atlas coordinate set to the patient coordinate set by performing a rigid registration between the at least one landmark identified in the patient coordinate set and the corresponding obtained atlas data in the atlas coordinate set and by performing a non-rigid registration of the three orders of division of the hepatic structure while maintaining the rigid registration of the at least one landmark.

### CROSS REFERENCE TO RELATED APPLICATIONS

This application is a continuation-in-part of currently pending U.S. patent application No. 13/234,903 filed Sept. 16, 2011, which is a continuation of prior filed International Application Serial Number PCT/US2010/027470 filed Mar. 16, 2010, which claims priority to U.S. provisional patent application No. 61/160,396 filed Mar. 16, 2009, all of which are hereby incorporated by reference into this disclosure.

### BACKGROUND OF THE INVENTION

Appreciation of the major features of hepatic anatomy was comprehensive by the 1960s, but only recently has a common terminology been adopted. Early anatomists recognized the lobularity of the hepatic surface; the right and left lobes were defined by the umbilical fissure and the attachment of the falciform ligament. While surface features were defined, understanding of the complex internal anatomy of the liver was limited. In 1897, James Cantlie used injection of vessels to define internal hepatic anatomy and suggested that the principal division of the liver is based on a vascular watershed, which lies in a plane intersecting the gallbladder fossa and the fossa for the inferior vena cava. Although the watershed is three-dimensional and therefore a plane, he called it a line which led to confusion.

In the 20^{th} century, Couinaud and Healey, independently used corrosion casting to define the intrahepatic divisions of blood vessels and bile ducts and thus established subdivisions of the liver. While their data was similar, each investigator anatomically divided the liver differently; Couinaud used the ramifications of the portal veins as the basis of division6 and Healey used the hepatic arteries and bile ducts.

Both investigators divided the liver into three orders or levels. Couinaud called the three levels of division: (1), livers or hemilivers (as in right and left livers); (2) sectors; and (3) segments. Healey called the three levels of division: (1) lobes; (2) segments; and (3) areas (or subsegments).

The difference between Couinaud and Healey's schemas rest in the left side of the liver, chiefly in the difference between the second-order divisions. Couinaud described the left portal vein to divide into an umbilical branch and a branch to segment 2. This divided the left liver into two sectors, consisting of a lateral sector (Couinaud segment 2) and a medial (or paramedian median) sector (Couinaud segments 3 and 4). Healey described the left hepatic artery and bile duct to divide into medial and lateral branches; the former supplied the part of the left liver to the right side of the umbilical fissure and the latter the part of the left liver to the left of the umbilical fissure. This divided the left liver into a medial segment (Couinaud segment 4) and a lateral segment (Couinaud segments 2 and 3). Lastly, Couinaud referred to the watersheds as scissuras (fissures or clefts) because, in his corrosion casts, such clefts were apparent.

Thus, hepatic terminology was left in a state of severe ambiguity. The confusion in anatomic terminology subsequently affected the terminology of liver surgery. Two kinds of lobectomy existed where some names for operations were based on surface anatomy and others were based on vascular watersheds.

In December 1998, the Scientific Committee of the International Hepato-Pancreato-Biliary Association created a terminology committee to deal with confusion in the nomenclature of hepatic anatomy and liver resections. This committee formulated a new terminology termed The Brisbane 2000 Terminology of Liver Anatomy and Resections which is anatomically and surgically correct, consistent, self-explanatory, linguistically correct, precise and concise.

The liver was divided into three functional livers: the right, the left and the caudate. The separation between the right and left hemiliver is at Cantlie's line, which is an oblique plane extending from the center of the gallbladder bed to the left bolder of the inferior vena cava. In this plane runs the middle hepatic vein, which is an important radiological landmark. The right hemiliver is divided further into two sections by the right portal scissura (anterior and posterior sections), within which runs the right hepatic vein. Each section is then divided on the basis of their blood supply and bile drainage into two segments. The anterior section is divided into segment 5 (inferior) and segment 8 (superior) and the posterior section into segment 6 (inferior) and segment 7 (superior)

The left hemiliver is divided into three segments. Segment 4 (quadrate lobe) is known as the left medial section, which lies to the right of the falciform ligament and its right margin forms the right margin of the left hemiliver. Segment 3, which lies in the anterior part, and segment 2, which lies in the posterior part of the left hemiliver, form the left lateral section. The left lateral section lies on the left of the falciform ligament. Between segment 2 and segment 3 runs the left hepatic vein. The caudate hemiliver (segment 1) is considered separately because of its separate blood supply, and venous and bile drainage.

The Brisbane 2000 Terminology of Liver Anatomy and Resections is the foundation for communication among hepatic surgeons. With the increasing use of stereotactic radiosurgery techniques by GI radiation oncologists, there is an increasing need for useful radiologic correlation with surgical anatomy.

### SUMMARY OF INVENTION

The invention includes, in a general embodiment, a method for the treatment of a hepatic tumor in a patient using radiotherapy. The method includes, providing a computer readable atlas of hepatic structure having a data set representing a three dimensional model of hepatic structure divided into three orders of division and comprising at least one of a plurality of hepatic anatomical structures and the relevant hepatic anatomy terminology.

The method further includes obtaining atlas data in an atlas coordinate set from the atlas and obtaining patient data in a patient coordinate set that corresponds to obtained atlas data in an atlas coordinate set, the patient data including the hepatic tumor. After the at last data and patient data are obtained, the method continues by identifying at least one landmark in the patient coordinate set, the at least one landmark corresponding to at least one of the plurality of hepatic anatomical structures of the atlas data.

The method continues by morphing atlas data from the atlas coordinate set to the patient coordinate set by performing a rigid registration between the at least one landmark identified in the patient coordinate set and the corresponding obtained atlas data in the atlas coordinate set and by performing a non-rigid registration of the three orders of division of the hepatic structure while maintaining the rigid registration of the at least one landmark. After the morphing is completed, the method continues by displaying an image of the patient coordinate set with the morphed atlas data including the three orders of division from the atlas for the treatment of the hepatic tumor using radiotherapy and providing the patient coordinate set and the morphed atlas data including the three orders of division from the atlas to a stereotactic radiation device for the treatment of the hepatic tumor.

In a preferred embodiment, the three orders of division include a first order separating the liver into two (right and left) hemi-livers, a second order transverse to the first order and dividing the liver into (anterior, posterior and medial) sections, and a third order (segments) transverse to the second order and approximately parallel to the first order and dividing the liver into segments. The first order of division is approximately correlated to Cantlie's Line. The second order of division is approximately correlated to the portal vein. The third order of division is approximately correlated to the hepatic vein. In total, these three orders of division divide the liver into 7 segments, with the left hemiliver divided into three segments and the right hemiliver is divided into 4 segments.

### BRIEF DESCRIPTION OF THE DRAWINGS

For a fuller understanding of the invention, reference should be made to the following detailed description, taken in connection with the accompanying drawings, in which:

FIG. 1: A diagram showing first-order division.

FIG. 2A: A diagram showing second-order division.

FIG. 2B: A diagram showing an alternate second-order division.

FIG. 3: A diagram showing second-order division.

FIG. 4: A diagram showing additional liver resections.

FIG. 5: A diagram illustrating the hepatic vascular anatomy.

FIGS. 6A and 6B: A diagram of the portal vein, defining the second order of division.

FIGS. 7A and 7B: A diagram of Cantlie's line (defining the first order of division) as well as thee left hepatic vein (defining one of the third order of division).

FIG. 8A-8C: A diagram showing the right hepatic vein defining the second of the the third order of division.

FIG. 9: A diagram (right anterior oblique view) showing segment 2, according to the three dimensional atlas.

FIG. 10: A diagram (right anterior oblique view) showing segment 3, according to the three dimensional atlas.

FIG. 11: A diagram (right anterior Oblique view) showing segments 4a and 4b, according to the three dimensional atlas.

FIG. 12: A diagram (right anterior oblique view) showing segment 5, according to the three dimensional atlas.

FIG. 13: A diagram (right anterior oblique view) showing segment 6, according to the three dimensional atlas.

FIG. 14: A diagram (right anterior oblique view) showing segment 7, according to the three dimensional atlas.

FIG. 15: A diagram (right anterior oblique view) showing segment 8, according to the three dimensional atlas.

FIG. 16: Right lateral view of a segmented liver according to the three dimensional atlas.

FIG. 17: Superior to inferior view of a segmented liver according to the three dimensional atlas.

### DETAILED DESCRIPTION OF THE PREFERRED EMBODIMENT

Many patients with liver metastases are not surgical candidates due to anatomic location or size of the tumor or medical inoperability. An important role exists for a treatment that can provide the equivalent of tumor excision, but with minimal morbidity. Recent technological advances have made it possible to deliver high doses of radiation therapy precisely to small tumors while preserving function in critical structures surrounding the lesion. Stereotactic conformal radiotherapy has been commonly used to treat small metastatic lesions in the brain. Recently, stereotactic liver radiation has been employed in the attempt to cure some patients unsuitable for resection.

Recently, radiation oncologists have developed a better understanding of the relationship between dose, volume of liver irradiated and radiation induced liver toxicity RILD). Based on an analysis of over 200 patients with hepatic malignancies, for a small effective liver volume irradiated, far higher doses of radiation can be prescribed than previously estimated. In addition to the dose and volume irradiated, several other factors were significantly associated with increased the risk of RILD, including use of BUdR chemotherapy (versus FuDR), primary hepatobiliary cancer diagnosis (versus metastatic cancer diagnosis) and male sex.

It has been suggested for patients with metastases, the mean liver dose associated with a 5% risk of RILD is 33 Gy in 2 Gy per fraction and 28 Gy in 10 fractions (assuming an alpha/beta ratio of 2.5 for the liver). It is well known that only a portion of the normal liver is required to sustain life. A surgical rule of thumb suggests that 25-40% of the normal liver must be spared during a resection. The Dose Volume Histogram (DVH) for the liver can be useful in quantifying the amount of liver receiving a “toxic” dose of radiation. At the University of Rochester, 17 initial patients were treated with 3D Conformal Therapy for primary or metastatic disease in the liver. The hepatic lesions ranged in size from 1-9.5 cm. Dose to the lesions ranged from 40-54 Gy using an average dose per fraction of 286 cGy (range 150-540 cGy). 60-70% of the non-tumor containing liver received a dose under 27-30 Gy. CT scans one month following radiation demonstrated focal liver changes within the treatment fields, Repeat CT scans three to six months later revealed measurable liver regeneration. None of the patients developed signs of liver failure or jaundice. Assessment of the anatomic region was not reported.

In patients with metastatic liver disease, aggressive local therapy using modern radiotherapy techniques should be studied in a multi-institutional setting to confirm safety and feasibility. Thus, a common system of reporting should be employed to facilitate multi-disciplinary and multi-institutional communication.

Different classification systems (Couinaud, Brisbane, Busmuth, Goldsmith and Woodburne for example) divide the liver into different sections/planes. American and French anatomists, for example, divide the left side through different planes. Such inconsistencies render surgical descriptions for hepatic resections unclear. The International Hepato-Pancreato-Biliary Association recommends the Brisbane 2000 system of liver anatomy. The Brisbane system is based on internal anatomy and includes three divisions: 1^{st} order (hemiliver), 2^{nd} order (sections) and 3^{rd} order (segments).

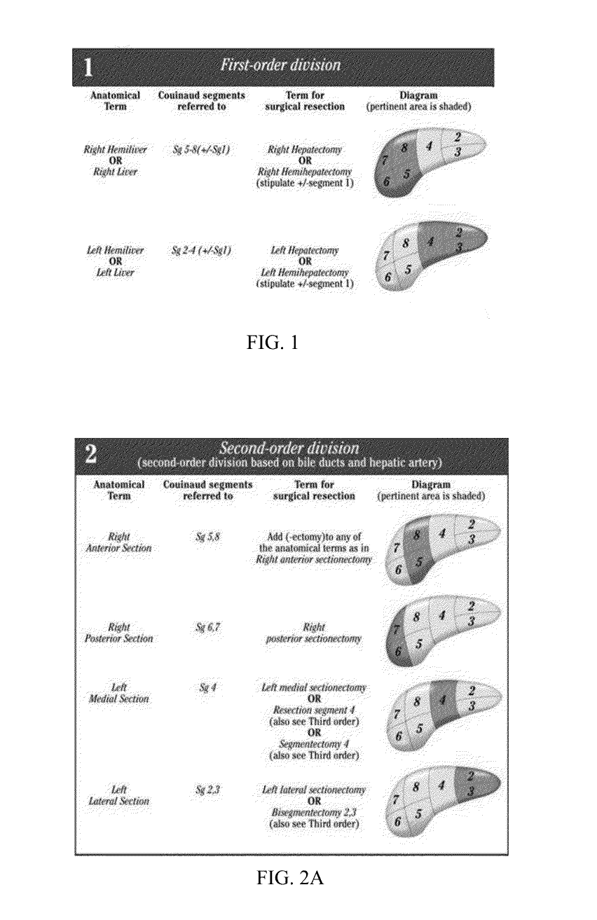

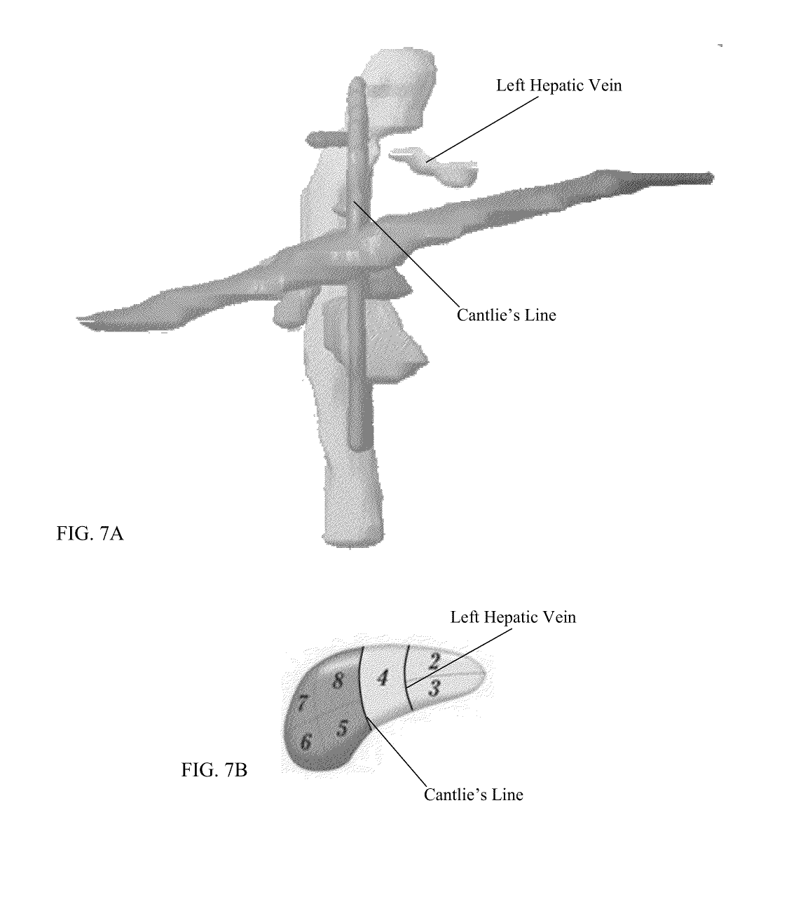

As shown in FIG. 1, the first order division includes the right hemiliver (right liver) or left hemiliver (left liver). The hemispheres are defined by the line between sections 4, and 8 and 5 along Cantlie's Line (FIG. 7A). Cantlie's Line is an extrapolated line from the posthepatic inferior vena cava across the diaphragmatic surface of the liver to the site where the fundus of the gallbladder typically contacts the inferior margin of the liver; approximates the anterior aspect of the plane of the middle hepatic vein and demarcates anteriorly the right and left (parts of the) liver(s),

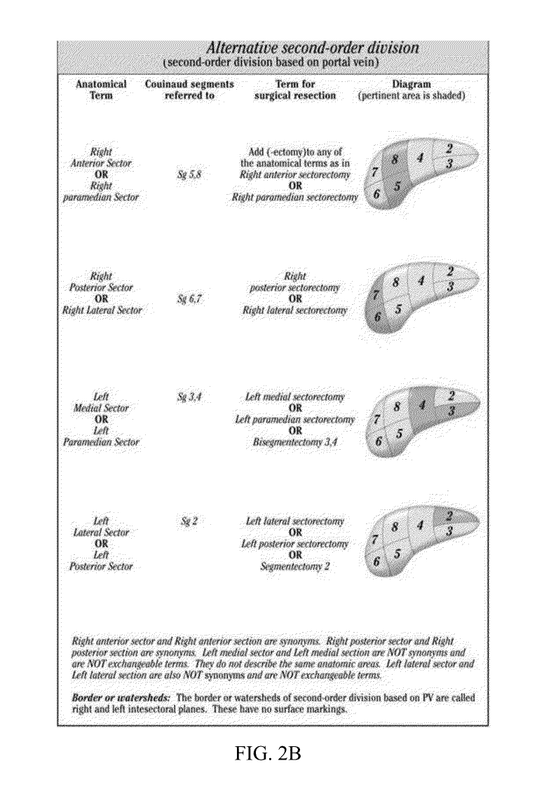

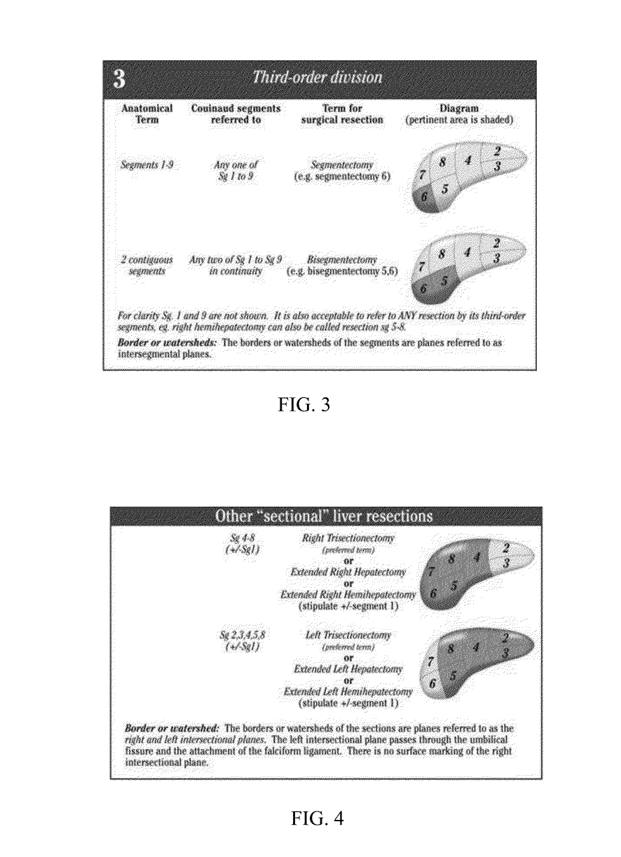

This border is a plane which intersects the gallbladder fossa and the fossa for the inferior vena cava. This is referred to the midplane of the liver. The second and third order divisions are shown in FIGS. 2-3 respectively. Additional liver resections are shown in FIG. 4.

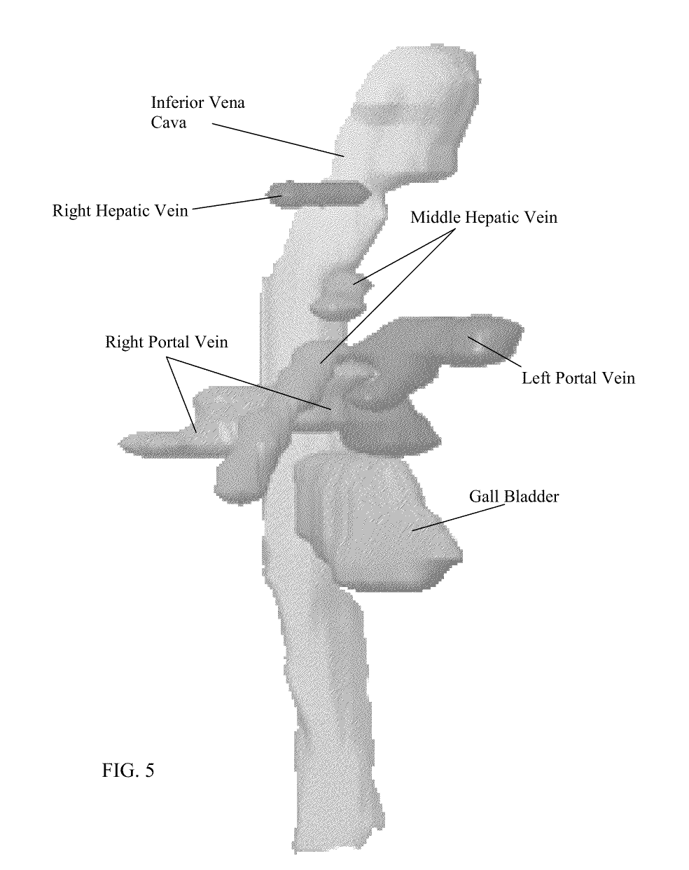

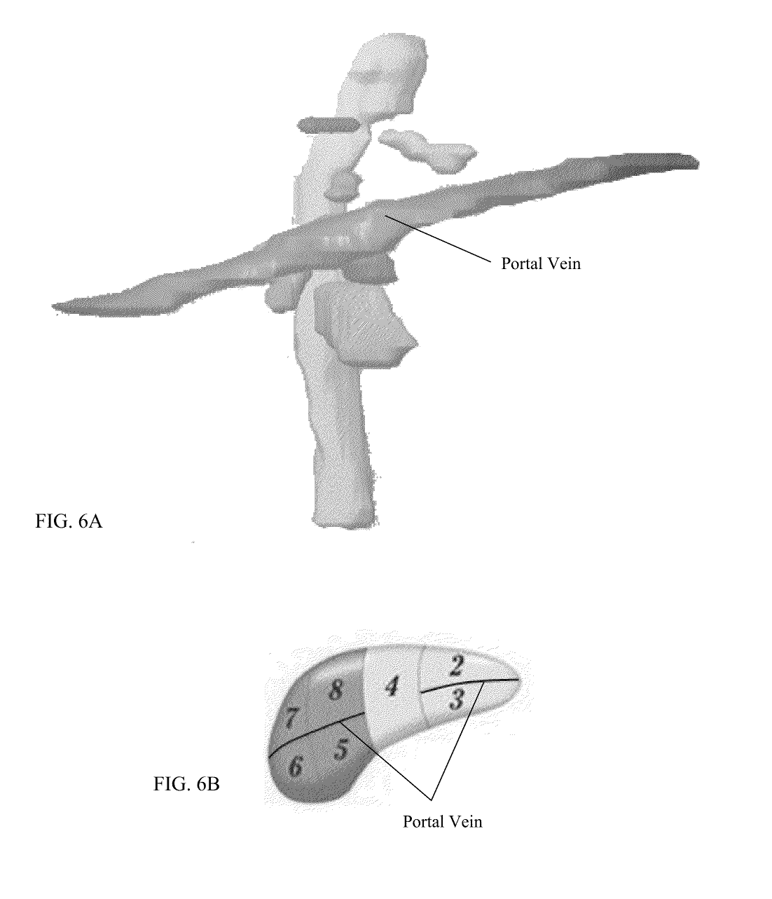

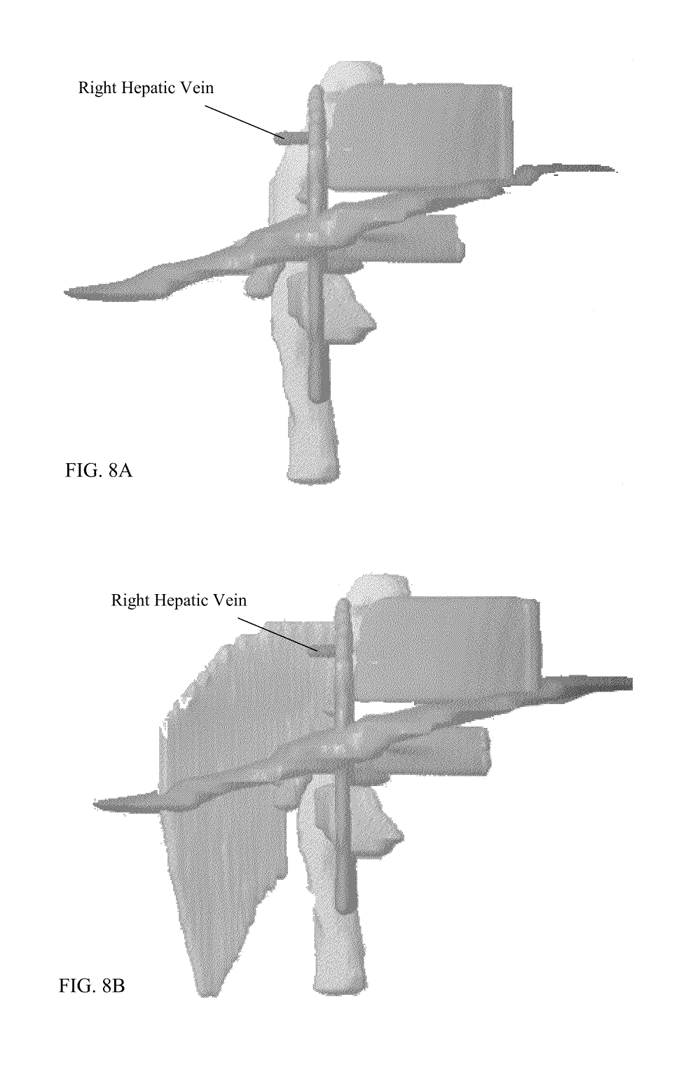

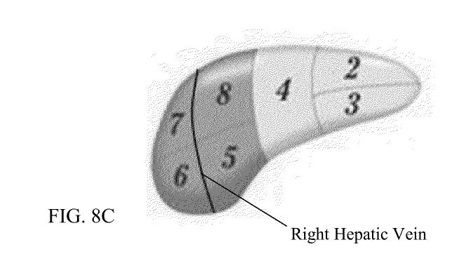

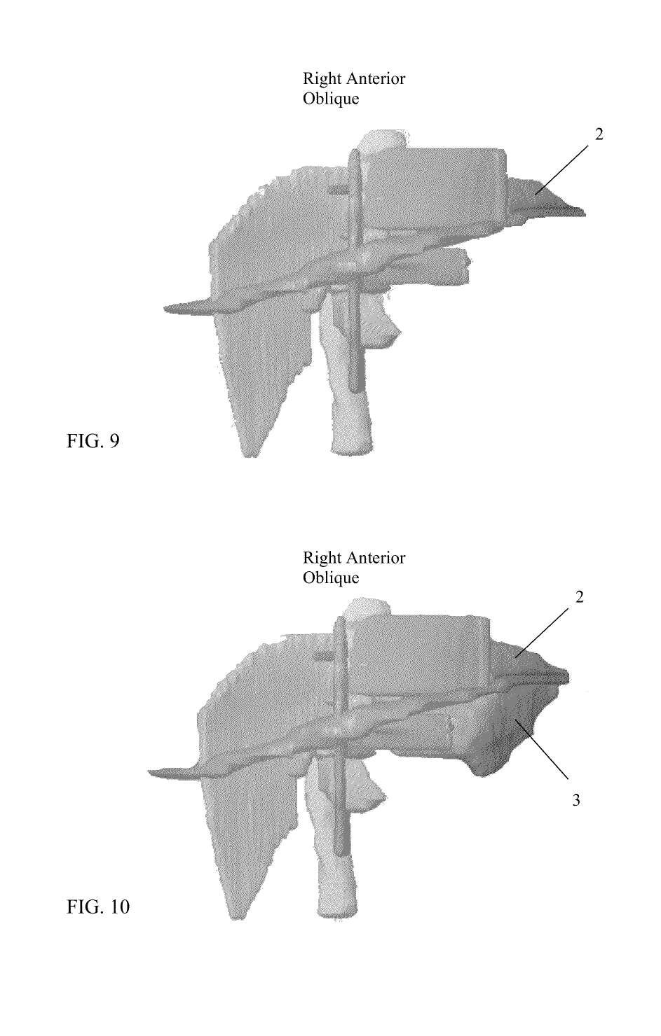

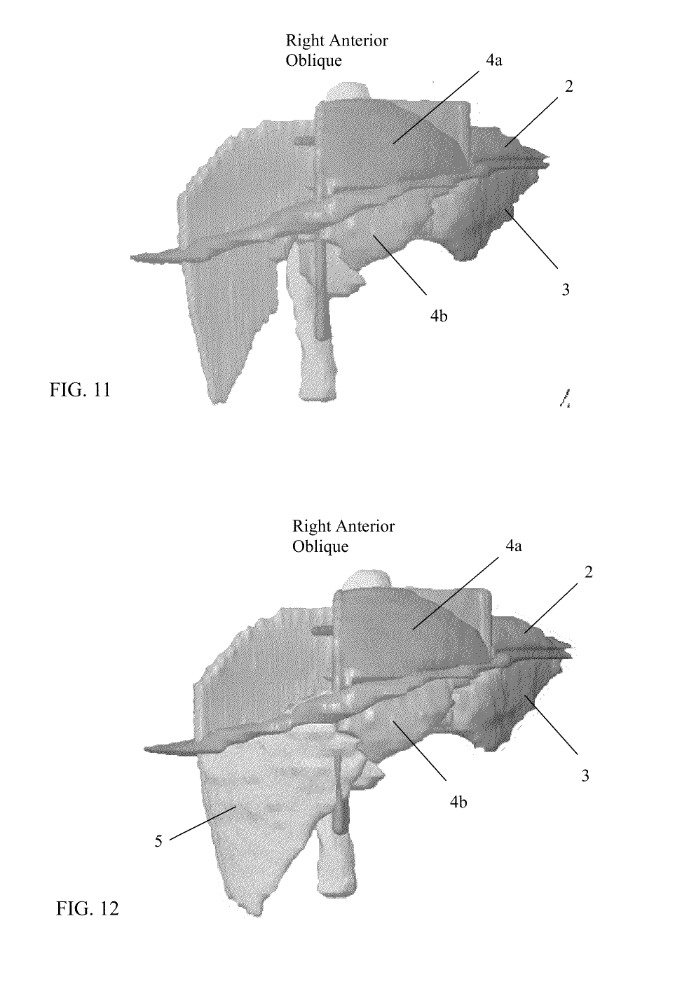

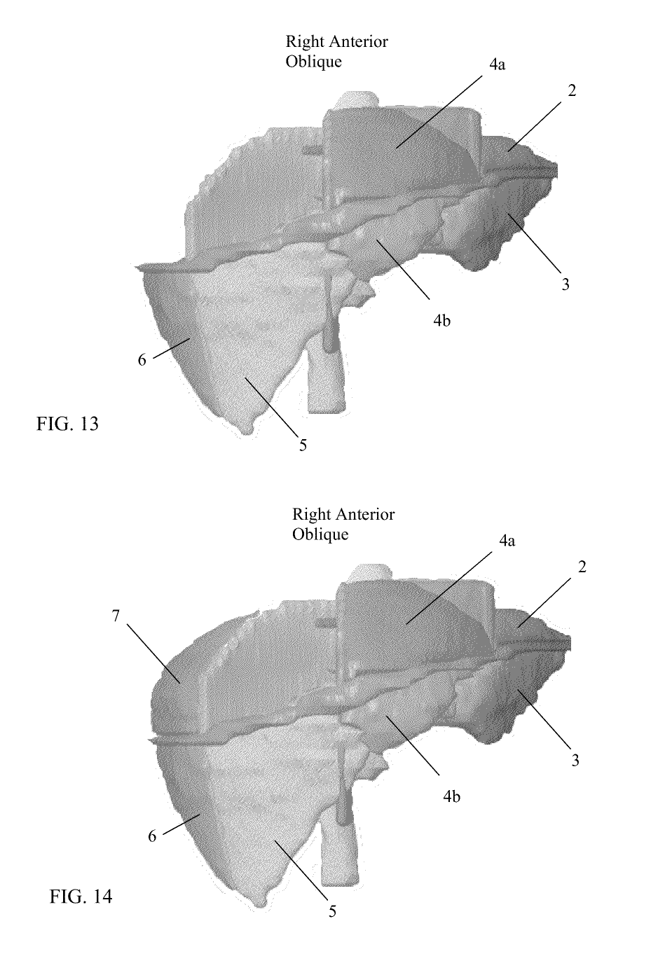

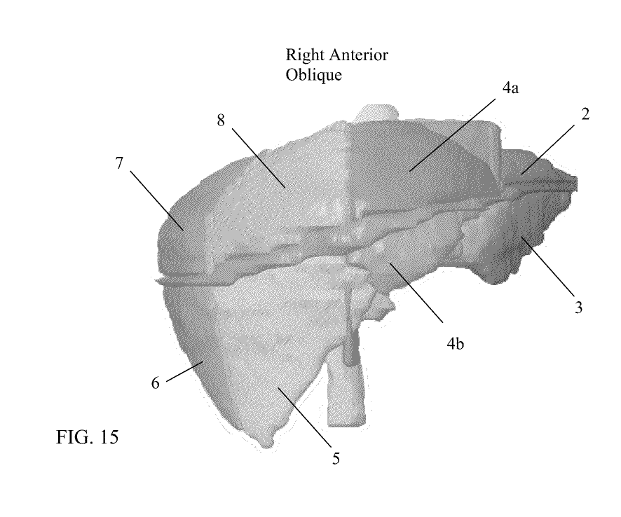

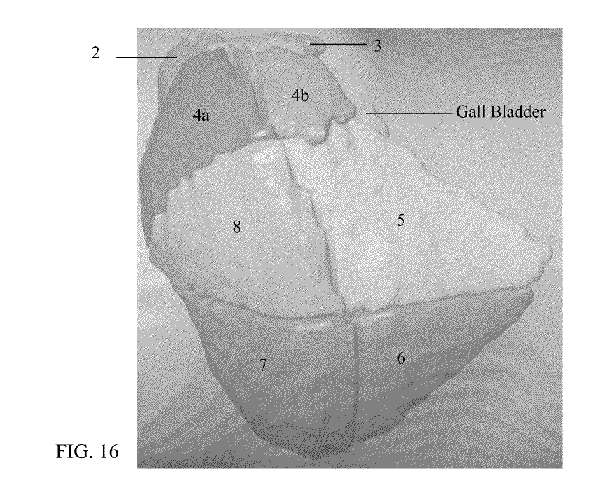

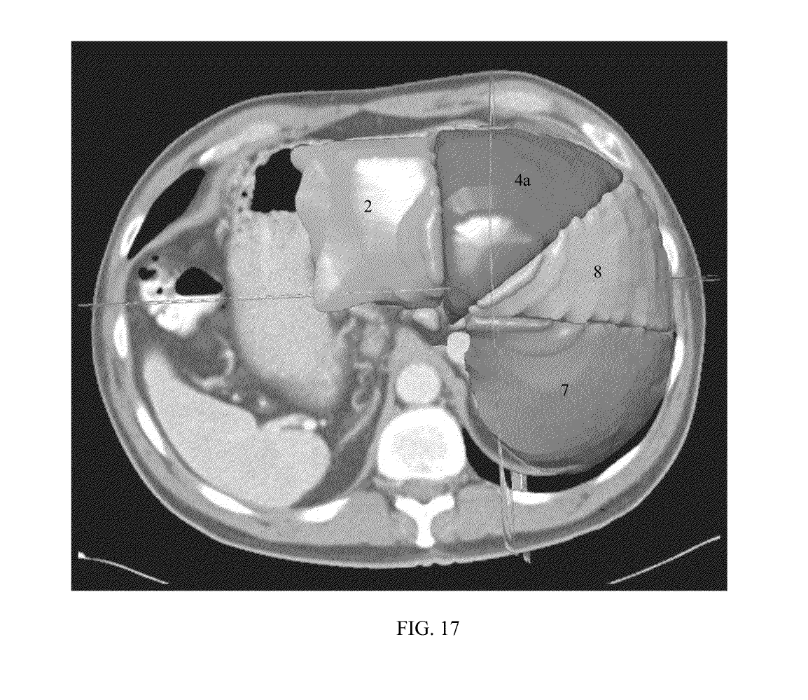

In developing the 3D atlas, the relevant hepatic vessels (inferior vena cava, left hepatic vein, left portal vein, middle hepatic vein, portal vein, right hepatic vein and right portal vein) were contoured. The relevant anatomic planes were inserted based on the vascular anatomy (FIGS. 5-8). The hepatic segments (3^{rd} order), numbered 1-8, lie between the planes (FIGS. 9-15). In this manner, the gross structure of hepatic anatomy is rendered (FIG. 16).

The second order division (FIGS. 2A and 2B) is based on bile ducts and the hepatic artery. An alternative second order division is based on the portal vein. The sections are referred to as right and left intersectoral planes and have no surface markings. The borders of the third order division are defined by the right and left hepatic vein (see FIGS. 3, 7 and 8 (descending portions omitted).

The invention also includes a method of deforming (transforming and/or morphing) data from the 3D atlas onto a diagnostic image of a subject of interest. In this way, the practitioner can easily correlate the anatomy of the subject, either in real life or on the diagnostic image, with the 3D atlas.

The methods and apparatuses described herein can improve the performance of interventions by taking advantage of transformations between the anatomy of an individual patient and an atlas. They can be useful in improving any of the four paradigms of intervention. The methods use both a rigid and a nonrigid, or deformable, transformation between the atlas and either the anatomy of an individual patient or one or more images of the anatomy of an individual patient, or a combination thereof. This can provide a physician with information otherwise unavailable.

An atlas is defined here, for the purpositions of this description, as a computer-readable description of anatomical information. The anatomical information may include images and geometrical entities and annotations and other information. An image may be: a one-dimensional image, such as an ultrasound echo or an X-ray line; a two-dimensional image, such as a plain X-ray image or an ultrasound image or a digitally reconstructed radiograph (DRR) formed from a three-dimensional image; a three-dimensional image, such as a computed tomography scan or a magnetic resonance image or a three-dimensional ultrasound image or a time sequence of two-dimensional images; or a four-dimensional image, such as a time sequence of three-dimensional images; or any other information that may be interpreted as an image. Geometrical entities may be: points; curves; surfaces; volumes; sets of geometrical entities; or any other information that may be interpreted as a geometrical entity. An annotation may be: material properties; physiological properties; radiological absorptiometric properties. An atlas, therefore, is a form of spatial database that can be queried and updated.

A transformation is a mathematical mapping of a point or an object in a first coordinate set C.sub.1 to a point or object in a second coordinate set C.sub.2. A transformation of a point can be represented as y=T(x) where x is a point in C.sub.1 and y is the point in C.sub.2 to which x is transformed. A transformation of every point in a first coordinate set to one or more points in a second coordinate set is a transformation from the first coordinate set to the second coordinate set. A transformation can be continuous or can be discontinuous. An invertible transformation is a transformation of a point in a first coordinate set C.sub.1 to a point in a second coordinate set C.sub.1, represented as y=T(x), such that there exists an inverse transformation x=T.sup.-1(y).

A parameterized transformation is a transformation in which mathematical entities called parameters take specific values; a parameter is a mathematical entity in the transformation other than the point in the first coordinate set that is transformed to a point in a second coordinate set so, for example, in the above definition of a rigid transformation both R and t are parameters of the rigid transformation. A parameter can vary continuously, in which case there are an infinite number of transformations specified by the parameter. A parameter can vary discretely, in which case there is a finite number of transformations specified by the parameter.

A morph is either an invertible deformable parameterized transformation or the result of applying an invertible deformable parameterized transformation to a set of points in a first coordinate set that maps to another set of points, whether in the same coordinate set or in a second coordinate set. Whether the term refers to the transformation itself, or to its application to a set of points, is understood from the context of usage by a practitioner of the art. In any embodiment the inverse of the deformable parameterized transformation may be found analytically or numerically or by any other means of inverting a transformation.

The method uses anatomical structures as points, or landmarks, for morphing the image from the 3D atlas. Illustrative structures include, but are not limited to, inferior hepatic angle, right portal vein bi-frication, hepatic-vena cava junction, medial angle of segment i, superior point of segment iii, apex of gallbladder (most superior aspect), on axial view, at plane level with medial angle of segment i, identify a selection of points outlining external liver volume, on coronal view, at plane level with medial angle of segment i, identify a selection of points outlining external liver volume.

In the present invention morphing atlas data from the atlas coordinate set to the patient coordinate set is accomplished by performing a rigid registration between the at least one landmark identified in the patient coordinate set and the corresponding obtained atlas data in the atlas coordinate set and by performing a non-rigid registration of the three orders of division of the hepatic structure while maintaining the rigid registration of the at least one landmark. Rigid registrations or transformation include rotations, translations, reflections, or their combination. In contrast, non-rigid registrations or transformations involve changes in the shape and/or size of a figure. As such, in accordance with the present invention, one or more landmarks of the hepatic structure are used to first perform a rigid registration between the atlas and the patient data and then the remaining, interval, hepatic structure is morphed using a non-rigid transformation between the atlas and the patient data.

The methods and apparatuses described herein use a morph or morphs for the purposition of providing computer-assisted intervention guidance. The methods and apparatuses are applicable to all four of the current paradigms for computer-assisted intervention, each of which will be described. The methods and apparatuses use morphing to establish a correspondence between an atlas and a patient, which is useful because information related to a geometric entity in the atlas can be related to the location of the morphed geometric entity in a patient coordinate set and, because of the invertibility of the morphing transformation, vice versa.

The use of morphing extends the preoperative-image paradigm by providing atlas information to the physician using the system. The atlas information is provided by morphing an atlas to the patient, or to a preoperative image, or to both, for the purposition of intraoperative guidance. The morphing transformation from the atlas to the patient can be calculated using data collected from the patient's anatomical surfaces, or data inferred from the patient's anatomy, or both forms of data, and data from the atlas. The morphing transformation from the atlas to a preoperative image can be calculated using data derived from the preoperative image and data from the atlas. The use of preoperative images in conjunction with the atlas can provide a better morph of the atlas to the patient.

Morphing for guidance using a preoperative image or images of a patient can be explained by way of an example of how knee surgery might be performed. Supposition that an atlas of the human left knee has been developed by merging several detailed scans of volunteer subjects by both computed tomography imaging and magnetic resonance imaging, with annotated information in the atlas provided by a practitioner skilled in the art of interpreting medical images. The annotations could include surface models of the bones, the mechanical center of the distal femur, the mechanical center of the femoral head, the mechanical axis that joins the centers, the transepicondylar axis, the insertion sites of the cruciate and collateral ligaments, the neutral lengths of the ligaments, and numerous other points and vectors and objects that describe clinically relevant features of the human left knee. Prior to surgery a preoperative CT image of the patient's right knee could be acquired by CT scanning The atlas images of the left knee could be morphed to the preoperative image of the patient's right knee by many means, such as point-based methods that minimize a least-squares disparity function, volumetric methods that maximize mutual information, or any other methods of determining a morphing transformation. The morph would need to include reflection about a plane to morph a left knee to a right knee, an example of such a plane being the sagittal plane.

During a surgical intervention, for example, a physician could determine a plurality of points on the surface of a patient's right femur, the points measured in a patient-based coordinate set. A registration transformation can then be calculated between the preoperative image and the points in a patient coordinate set, such that a disparity function of the points and the surface models is minimized. The morph transformation from an atlas coordinate set to the preoperative image can then be compositional with the registration transformation to provide a morph transformation from an atlas coordinate set to a patient coordinate set. Using the morph transformation, a point in an atlas coordinate set can be morphed into a patient coordinate set. The morphed point can be used in many ways, such as to determine the distance of the morphed point from one of the annotated axes, which provides to a physician an estimate of the location of an axis in a patient where the axis might be difficult to estimate directly from the patient. A computer program can then provide to the physician images derived from the preoperative image, and images and annotations derived from the atlas, to improve the physician's ability to plan and perform the surgical procedure.

In an illustrative embodiment for providing interventional guidance with preoperative images of a patient, a computer program communicates with a tracking system and can access one or more preoperative images and an atlas. A preferred embodiment utilizes a configuration having a first tracked device 401 a with coordinate set is attached to a patient and a tracking system provides to a computer program in computer the position of the first tracked device, in the preferred embodiment position is in the coordinate set of the first tracked device. In an alternative embodiment this position is provided in a second coordinate set. A second tracked device is attached to an actual instrument. In the preferred embodiment the position of the second tracked device with coordinate set is provided to the computer program in coordinate set of the first tracked device. In an alternative embodiment the position of the tracked device is provided to the computer program in the second coordinate set and the computer program computes the relative position of the second tracked device with respect to the coordinate set of the first tracked device.

As a physician directly contacts surfaces of anatomical regions of the patient and the tracking system, or the computer program, or both, can determine the position of the guidance point on the actual instrument in the coordinate set of the first tracked device, so that the coordinate set of the first tracked device acts as the coordinate set of the patient.

A method, additionally embodied in the computer program, is shown that can be used for morphed guidance with an atlas image, in which the morph transformation from the atlas coordinate set to the patient coordinate set and position of the tracked actual instrument from the coordinate set relative to the patient coordinate set can be combined with a morph or registration transformation from a coordinate set of a preoperative image.

A morph transformation and tracking of the actual instrument can be used to morph an atlas image and superimposition an image of a virtual instrument on a morphed slice of the atlas image, in combination or separate from use of a registration transformation and tracking of the actual instrument can be used to show a preoperative image and to superimposition an image of a virtual instrument on a morphed slice of the preoperative image.

In the preferred embodiment of the computer program one or more morph transformations are calculated from the coordinate set or sets of the atlas to the coordinate set or sets of the preoperative image or images. A parameterization of a rigid transformation from the coordinate set of a preoperative image to the coordinate set of the patient is formulated. The parameters of the rigid transformation are calculated so as to minimize a disparity function between the translated data in the preoperative image and corresponding data in the patient coordinate set. The resulting registration can be mathematically and numerically compositional with a morph from an atlas coordinate set to a preoperative-image coordinate set and thus provide a morph from an atlas coordinate set to the patient coordinate set.

Preferred embodiments can include coordinate transformations in which registration transformation from a coordinate set of a preoperative image to coordinate set of the patient is calculated from patient data, and morph transformation from a coordinate set of an atlas to a coordinate set of a preoperative image is calculated from image data, and morph transformation from a coordinate set of an atlas to coordinate set of the patient is compositional from the other two transformations, and relative position of the coordinate set of a tracked actual instrument is provided from information provided by a tracking system. By means of these calculations the method provides morphs from an atlas to a patient and morphs from an atlas to a preoperative image, as well as registrations from a preoperative image to a patient.

In a first alternative embodiment for providing interventional guidance with preoperative images of a patient, the surface points in the patient coordinate set are used as data to determine one or more rigid transformations between the coordinate set or sets of the preoperative image or images and the patient coordinate set. The patient data are also used to determine one or more morph transformations from the coordinate set or sets of the atlas to the patient coordinate set.

The coordinate transformations of the first alternative embodiment include registration transformation from a coordinate set of a preoperative image to coordinate set of the patient is calculated from patient data and morph transformation from a coordinate set of an atlas to a coordinate set of a preoperative image is calculated from image data and morph transformation from a coordinate set of an atlas to coordinate set of the patient is calculated from patient data and relative position of the coordinate set of a tracked actual instrument is provided from information provided by a tracking system. By means of these calculations the method provides morphs from an atlas to a patient and morphs from an atlas to a preoperative, as well as registrations from a preoperative image to an atlas.

In a second alternative embodiment for providing interventional guidance with preoperative images of a patient, one or more morph transformations are calculated from the coordinate set or sets of the atlas to the coordinate set or sets of the preoperative image or images. In the second alternative embodiment the surface points in the patient coordinate set are used as data to determine one or more morph transformations from the coordinate set or sets of the atlas to the patient coordinate set,

The coordinate transformations of the second alternative embodiment in which morph transformation from a coordinate set of an atlas to a coordinate set of a preoperative image is calculated from image data and morph transformation from a coordinate set of an atlas to coordinate set of the patient is calculated from patient data and morph transformation from a coordinate set of a preoperative image to coordinate set of the patient is calculated from the other two transformations and relative position of the coordinate set of a tracked actual instrument is provided from information provided by a tracking system. By means of these calculations the method provides morphs from an atlas to a patient and morphs from an atlas to a preoperative image and morphs from a preoperative image to a patient.

In a third alternative embodiment for providing interventional guidance with preoperative images of a patient, the surface points in the patient coordinate set are used to determine one or more rigid transformations between the coordinate set or sets of the preoperative image or images and the patient coordinate set. The surface points data are also used to determine one or more morph transformations from the coordinate set or sets of the atlas to the patient coordinate set. The resulting registration can be mathematically and numerically compositioned with a morph from an atlas coordinate set to the patient coordinate set and thus provide a morph from an atlas coordinate set to a preoperative-image coordinate set.

The coordinate transformations of the third alternative embodiment include registration transformation from a coordinate set of a preoperative image to coordinate set of the patient is calculated from patient data and morph transformation from a coordinate set of an atlas to coordinate set of the patient is calculated from patient data and morph transformation from a coordinate set of an atlas to a coordinate set of a preoperative image is calculated from the other two transformations and relative position of the coordinate set of a tracked actual instrument is provided from information provided by a tracking system. By means of these calculations the method provides morphs from an atlas to a patient and morphs from an atlas to a preoperative image, as well as registrations from a preoperative image to a patient.

In a fourth alternative embodiment for providing interventional guidance with preoperative images of a patient, the surface points in the patient coordinate set are used as data to determine one or more rigid transformations between the coordinate set or sets of the preoperative image or images and the patient coordinate set. The surface data are also used to determine one or more morph transformations from the coordinate set or sets of the atlas to the patient coordinate set.

The coordinate transformations of the fourth alternative embodiment include registration transformation from a coordinate set of a preoperative image to coordinate set of the patient is calculated from patient data and morph transformation from a coordinate set of an atlas to coordinate set of the patient is calculated from patient data and relative position of the coordinate set of a tracked actual instrument 404d is provided from information provided by a tracking system. By means of these calculations the method provides morphs from an atlas to a patient and registrations from a preoperative image to a patient.

In a fifth alternative embodiment for providing interventional guidance with preoperative images of a patient, one or more morph transformations are calculated from the coordinate set or sets of the atlas to the coordinate set or sets coordinate set of the preoperative image or images. In the fifth alternative embodiment the surface points in the patient coordinate set are used as data to determine one or more morph transformations from the coordinate set or sets of the atlas to the patient coordinate set.

The coordinate transformations of the fifth alternative embodiment include morph transformation from a coordinate set of an atlas to a coordinate set of a preoperative image is calculated from image data and morph transformation from a coordinate set of an atlas to coordinate set of the patient is calculated from patient and relative position of the coordinate set 402 of a tracked actual instrument is provided from information provided by a tracking system. By means of these calculations the method provide morphs from an atlas to a patient and morphs from an atlas to a preoperative image.

The computer program, or another computer program, can subsequently relate the location of the tracked actual instrument or of another tracked actual instrument to the atlas. In the preferred embodiment, the computer program morphs images and other atlas data to the coordinate set of the patient, and displays these images and data to the physician with a computer representation of the tracked actual instrument superimpositioned upon these images and data. By this method the physician can use the images and data to guide a tracked actual instrument within the patient's body. In an alternative embodiment, the computer program morphs the coordinate set of the patient to the coordinate set or sets of the atlas by means of the inverse of the morph transformation from the atlas coordinate set or sets to the patient coordinate set, and displays atlas images and data to the physician with a computer representation of the deformed tracked actual instrument superimpositioned upon these images and data.

Other data determined in the coordinate set of the patient can be used to morph an atlas to a patient, as described in the use of the preferred embodiment for guidance without images. A morphing transformation can be used to provide atlas data to an interventionalist, as described in the use of the preferred embodiment for guidance without images.

The present invention may be embodied on various computing platforms that perform actions responsive to software-based instructions. The following provides an antecedent basis for the information technology that may be utilized to enable the invention.

The computer readable medium described in the claims below may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wire-line, optical fiber cable, radio frequency, etc., or any suitable combination of the foregoing. Computer program code for carrying out operations for aspects of the present invention may be written in any combination of one or more programming languages, including an object oriented programming language such as Java, C#, C++ or the like and conventional procedural programming languages, such as the “C” programming language or similar programming languages.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a

particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

It will be seen that the advantages set forth above, and those made apparent from the foregoing description, are efficiently attained and since certain changes may be made in the above construction without departing from the scope of the invention, it is intended that all matters contained in the theregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

What is claimed is:

1. A method for the treatment of a hepatic tumor in a patient using radiotherapy, the method, comprising:
providing a computer readable atlas of hepatic structure having a data set representing a three dimensional model of hepatic structure divided into three orders of division and comprising at least one of a plurality of hepatic anatomical structures and the relevant hepatic anatomy terminology;
obtaining atlas data in an atlas coordinate set from the atlas;
obtaining patient data in a patient coordinate set that corresponds to obtained atlas data in an atlas coordinate set, the patient data including the hepatic tumor;
identifying at least one landmark in the patient coordinate set, the at least one landmark corresponding to at least one of the plurality of hepatic anatomical structures of the atlas data;
morphing atlas data from the atlas coordinate set to the patient coordinate set by performing a rigid registration between the at least one landmark identified in the patient coordinate set and the corresponding obtained atlas data in the atlas coordinate set and by performing a non-rigid registration of the three orders of division of the hepatic structure while maintaining the rigid registration of the at least one landmark;
displaying an image of the patient coordinate set with the morphed atlas data including the three orders of division from the atlas fir the treatment of the hepatic tumor using radiotherapy; and
providing the patient coordinate set and the morphed atlas data including the three orders of division from the atlas to a stereotactic radiation device for the treatment of the hepatic tumor.

2. The method of claim 1, wherein the step of obtaining patient data in a patient coordinate set that corresponds to atlas data in an atlas coordinate set comprises the step of, collecting a plurality of points in a patient coordinate set from the patient that correspond to points in an atlas coordinate set from the atlas.

3. The method of claim 1, wherein the obtained patient data comprises a plurality of points from the patient anatomy in a patient coordinate set, and the obtained atlas data comprises a plurality of points from the atlas in an atlas coordinate set.

4. The method of claim 1, wherein the step of obtaining a plurality of points in a patient coordinate set that correspond to points in an atlas coordinate set from the atlas comprises:
obtaining an image of the patient including a plurality of points in an image coordinate set that correspond to points in an atlas coordinate set from the atlas; and
collecting a plurality of points in a patient coordinate set from the patient that correspond to points in an atlas coordinate set from the atlas; and collecting a plurality of points in a patient coordinate set from the patient that correspond to points in an image coordinate set from the image,

5. The method of claim 1, wherein the three orders of division include a first order of division separating the liver into two hemilivers, a second order of division transverse to the first order of division and dividing the liver into sections, and a third order of division transverse to the second order of division and approximately parallel to the first order of division and dividing the liver into segments.

6. The method of claim 5, wherein the first order of division is approximately correlated to Cantlie's Line.

7. The method of claim 5, wherein the second order of division is approximately correlated to the portal vein.

8. The method of claim 5, wherein the third order of division is approximately correlated to the hepatic vein.

9. The method of claim 6, wherein the three orders of division divide the liver into 7 segments.

10. The method of claim 5, wherein the left hemiliver is divided into three segments and the right hemiliver is divided into 4 segments,

11. The method of claim 1, wherein the at least one of a plurality of anatomical structures is selected from the group consisting of, inferior hepatic angle, right portal vein bi-frication, hepatic-vena cava junction, medial angle of a segment, superior point of a segment, apex of gallbladder, a selection of points outlining external liver volume.

12. The method of claim 1, wherein the data set further comprises the radiological absorptiometric properties of the hepatic anatomy.

13. The method of claim 1, wherein the computer readable atlas is representative of a Brisbane 2000 Terminology of Liver Anatomy and Resections.

14. One or more non-transitory computer-readable media having computer-executable instructions for performing a method of running a software program on a computing device for the treatment of a hepatic tumor in a patient using radiotherapy, the computing device operating under an operating system, the method including issuing instructions from the software program comprising:
providing a computer readable atlas of hepatic structure having a data set representing a three dimensional model of hepatic structure divided into three orders of division and comprising at least one of a plurality of hepatic anatomical structures and the relevant hepatic anatomy terminology;
obtaining atlas data in an atlas coordinate set from the atlas;
obtaining patient data in a patient coordinate set that corresponds to obtained atlas data in an atlas coordinate set, the patient data including the hepatic tumor;
identifying at least one landmark in the patient coordinate set, the at least one landmark corresponding to at least one of the plurality of hepatic anatomical structures of the atlas data;
morphing atlas data from the atlas coordinate set to the patient coordinate set by performing a rigid registration between the at least one landmark identified in the patient coordinate set and the corresponding obtained atlas data in the atlas coordinate set and by performing a non-rigid registration of the three orders of division of the hepatic structure while maintaining the rigid registration of the at least one landmark;
displaying an image of the patient coordinate set with the morphed atlas data including the three orders of division from the atlas for the treatment of the hepatic tumor using radiotherapy; and
providing the patient coordinate set and the morphed atlas data including the three orders of division from the atlas to a stereotactic radiation device for the treatment of the hepatic tumor.

15. The media of claim 14, wherein the step of obtaining patient data in a patient coordinate set that corresponds to atlas data in an atlas coordinate set comprises the step of, collecting a plurality of points in a patient coordinate set from the patient that correspond to points in an atlas coordinate set from the atlas.

16. The media of claim 14, wherein the obtained patient data comprises a plurality of points from the patient anatomy in a patient coordinate set, and the obtained atlas data comprises a plurality of points from the atlas in an atlas coordinate set.

17. The media of claim 14, wherein the step of obtaining a plurality of points in a patient coordinate set that correspond to points in an atlas coordinate set from the atlas comprises:
obtaining an image of the patient including a plurality of points in an image coordinate set that correspond to points in an atlas coordinate set from the atlas; and
collecting a plurality of points in a patient coordinate set from the patient that correspond to points in an atlas coordinate set from the atlas; and collecting a plurality of points in a patient coordinate set from the patient that correspond to points in an image coordinate set from the image.

18. The media of claim 14, wherein the three orders of division include a first order of division separating the liver into two hemilivers, a second order of division transverse to the first order of division and dividing the liver into sections, and a third order of division transverse to the second order of division and approximately parallel to the first order of division and dividing the liver into segments.

19. The media of claim 14, wherein the at least one of a plurality of anatomical structures is selected from the group consisting of, inferior hepatic angle, right portal vein bi-frication, hepatic-vena cava junction, medial angle of a segment, superior point of a segment, apex of gallbladder, a selection of points outlining external liver volume.

20. The media of claim 14, wherein the data set further comprises the radiological absorptiometric properties of the hepatic anatomy.
